(19) United States
(12) Patent Application Publication
(10) Pub. No.: US 2016/0158395 A1
(43) Pub. Date: Jun. 9, 2016

(54) UVC Sterilization Box Electronics Devices

(71) Applicants: Kenneth Gregory Sweeney, Oceanside, CA (US); Louis Sylvester Hughes, Rancho Santa Fe, CA (US)

(72) Inventors: LOUIS SYLVESTER HUGHES, San Diego, CA (US); KENNETH GREGORY SWEENEY, OCEANSIDE, CA (US)

(21) Appl. No.: 14/217,482

(22) Filed: Mar. 18, 2014

(51) Int. Cl. A61L 2/10

(52) U.S. Cl. CPC A61L 2/10

(57) ABSTRACT

A sterilization method and apparatus for cleaning multiple devices of different sizes on all surfaces at one time. This apparatus allows for the cleaning of electronic devices which are corded and cordless/wireless without allowing ozone which is generated from the UV lights to escape from the product or allowing humans eyes to be effected by direct exposure to the UV light generated by the apparatus. This apparatus enables devices to be charged while they are being sterilized. This invention outlines a very simple method for replacement of fragile cold cathode lamps using a wire harness and metalized enclosure

### CROSS-REFERENCE TO RELATED APPLICATIONS

Provisional Patent Application No. 61/792,673 Dated Mar. 15, 2013 Attached hereto

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### FIELD OF INVENTION

This invention relates to the field of germicidal sterilization or sanitizing of device and/or product surfaces.

### BACKGROUND OF THE INVENTION

Portable hand held electronics devices like cell phones, cordless phones, remote controls and keyboards are known carriers of bacteria and viruses. They are this way because people touch them often with their hands which they use to touch many other things which carry bacteria. There are specific markets like healthcare where the distribution of multi drug resistant (MDRO) bacteria is a life and death matter.

Today, hospital acquired infections are incurred by one in 20 patients admitted to the hospital (affecting 1.7 million patients each year). The cost is enormous, adding more than $40 billion to the bottom line for healthcare in the US alone. Antibiotic resistant infections due to “Superbugs” are on the rise. One superbug, called “MRSA,” affecting over 100,000 patients a year, caused the death of more than 28,600 patients in 2009. This number supersedes the death rate for breast cancer, AIDS and SARS combined. The spread of hospital acquired infections is an increasing problem in many hospitals and clinics throughout the world. Anti-biotic resistant infections such as MRSA and C. Difficile are all too common and result in thousands of deaths each year. Controlling infection throughout hospitals is an important aspect of good clinical management and many measures have been introduced to limit the spread and protect the vulnerable in our hospital wards. Barrier creams and enforced hand washing are only part of the story in helping to eliminate hospital acquired infections as there are other measures that need to be considered too. Preventing the spread of infection is not just about keeping the wards clean.

Commonly, in modern healthcare settings, the patient has access to a phone and to a separate “all in one” remote control/speaker/nurse call button device that has a cord. This device is handled by the patient themselves, the nurse, doctor, and visitors alike. Additionally, patients have access to their personal mobile phone as well. All of these devices are typically not cleaned in such a way to eliminate the germs and other bacteria. They are difficult, if not impossible to clean based on them being sensitive or vulnerable to liquids and to the harsh chemical used for cleaning these devices. The design of most remotes, phones, and other mobile devices means there are too many nooks and crannies for bacteria and pathogens to linger.

The current solutions for cleaning these devices are through the use of chemicals and detergents. Washable and medical remote controls are now available which remove the nooks and crannies and provide a waterproof barrier for the electronics. The problem is that these solutions still need to be cleaned on an hourly or per shift basis. Because the chemicals need to be left on the device “wet” for some period of time to clean these aggressive bugs away, the cleaning process is still time consuming and labor intensive. The chemicals that must be used are harmful to the environment. Because the cleaning process is time consuming, the hospitals have a huge problem with compliance to the cleaning regimens. If something is difficult and time consuming to do some people will not do it. All it takes is a few people not complying with washing and the bacteria distribution network starts.

Our present inventions leverage the use of a 100 year old technology called ultra violet light at the 254 nanometer wavelength. The anti-microbial properties of UV-C light are well known to scientists and have been used since the 1930's to kill germs containing DNA and RNA (including bacteria, viruses, fungi and mold). UV-C light is invisible to the human eye. The ultraviolet light which the devices in this invention are using is in the germicidal wavelength—200-280 nanometers—which renders the organisms sterile. When organisms can no longer reproduce, they die. UV-C light is used in many anti-bacterial and anti-microbial applications today including in health/sports clubs, professional sports locker rooms, food processing plants, water purification systems, personal hygiene consumer products, in hand held generic wands and is generally harmless to the human body in applications at 15 watts and under.

We have taken this UVC light and have applied it to new devices which make it simple and convenient to clean multi touch electronic devices. The market requirement is to provide a Portable Electronic cleaning solution which enables the cleaning of the entire current Portable Electronic device found in the patient's rooms today. For convenience, the devices need to be able to be cleaned with a wire connected to them since hospital devices like remote controls and phones are “wired” devices having a cable that goes from the unit to the wall or the bed. You must be able to insert your devices, and allow the cable to come out of the device WHILE it is being sterilized. We have accomplished that with our design. In addition, having the ability to charge your mobile phone device is an added benefit and feature that is both efficient and simple to use. While your device is being sterilized, there is an AC outlet designed into our solution that allows you to charge your mobile device during the cleaning cycle. There is no UVC Portable Electronics cleaning device either contemplated or designed which performs all of the above critical functions; cleaning wired remote controls, small in size to be installed on a wall or bedside table, allows charging of your device while it is being sterilized, insures compliance of the cleaning procedures for devices in the room, and is easy to use.

### SUMMARY OF THE INVENTION

The present invention includes a new cleaning and sterilization device for remote controls, mobile phones, portable media players, eBook readers and even car keys! This device uses UVC light to kill bacteria and viruses. This device uses low wattage cold cathode UVC bulbs due to the small area available for the bulb. Cold cathode light bulbs are very fragile and are very difficult to handle directly without cracking them or getting hand prints on them. Previous designs of cold cathode based UVC sterilization boxes require the cold cathode bulbs to be handled directly when the bulb is changed out. This is very difficult when the bulbs need to be changed out by people that are not familiar with handling a cold cathode bulb. This new and innovative solution packages the UVC bulbs in a simple, two-part metalized plastic shell which is snapped together and slid into the cleaning box. The entire package slides along the length or height of the UVC cleaning box. There is a simple connector that is attached to the box after it is slid into place and this same connector is disconnected when the bulb assembly is removed. This new UVC bulb cold cathode packaging technique ensures that the cold cathode bulb is never touched directly by human hands when the bulb is being replaced.

The unit has a built in timer that allows the unit to stay on for a predetermined number of minutes to insure the portable electronic devices inside are sanitized/sterilized correctly and completely each and every time. The timer starts when the covered is closed and runs automatically for a set period cycle. The unit shuts off if the door is opened.

Many people or users wish to sterilize their battery powered portable electronic devices while they re-charge them. When they put their portable electronic devices inside a UVC or other type of electronic sterilization device, they would like to be able to charge them at the same time. The problem is that there is not always a power source within reach of the sterilization device. This is especially true in hospitals where the power plugs are not within reach of a patient in a bed (they are too far down close to the floor). The new sterilization or cleaning system includes a power outlet on the side of the box so that when the battery powered portable electronic device is being cleaned it can also be recharged. When the patient or user puts their portable battery powered device in the sterilization box they just plug the device right into the power plug on the side of the box.

### BRIEF DESCRIPTION OF THE DRAWINGS

FIG. 1a: Shows the Cold Cathode UVC bulb 101 inside the two part plastic, metalized package being described in this disclosure 102. There is a front half which is a grill part 106 which is snapped into the rear part. The rear part is metalized and acts as a reflector to push UVC light and sterilization effect back into the box to sterilize components inside it. 100 is the main sterilization box.

FIG. 2: View of the area built into Sterilization box where the Cold Cathode UVC assembly is inserted. The shows the two part plastic package 102 and the cold cathode UVC bulb inside it 101 and how it slides into the sterilization box on the front side and rear side of the box 100. This is critical because you need to have UVC light hitting the front and back of any device being sterilized.

FIG. 3: Shows the view of the Sterilization box from the top down. Shows the cold cathode UVC bulb package installed into the sterilization box. The package provides protection for the cold cathode UVC bulb BOTH during the changing of the bulb AND during the use of the sterilization device/box when the UVC bulb is being operated for sterilization.

FIG. 4: Shows the simple snap fit covers 105 that are used on the bottom of the Sterilization box 100 to hold the Cold Cathode UVC bulb assembly into place inside the box for operation. These simple snaps are removed when the cold cathode UVC bulb assembly needs to be replaced. There are no bulb sockets or bulb bases to worry about in this unique and innovative solution

FIG. 5: Shows a broader view of the sterilization box and the location where the cold cathode UVC bulb assembly is installed inside the unit

FIG. 6: Shows a view of the hospital room environment. The wall of the hospital patient room 110 right next to the bed of the patient has many devices hanging off of it for patient care 108. The depth of these patient care devices is measured by our ruler 109 to be less than 6 inches.

FIG. 7: Shows cut out view of the “second” platform 112 where the smaller electronic devices can be sterilized for easy access in and out of the box. There are two levels for sterilization, a “first” level which is deep and provides enough room to sterilize a remote control or corded phone and a “second” level 112 which is shorter and sterilizes smaller devices like mobile phones and keys. There is a fence in place 111 along the width of this shelf that prevents devices from slipping off the shelf while allowing the UV light to pass through.

FIG. 8: Shows a view of the actual sterilization box product with the two different electronics devices inside stored on the two different sterilization platforms (112,113). If there was no “second” platform 112 then the smaller portable devices and smaller products like car keys could not be reached with the human hand. This design must be hung on the wall and for that reason the design must not have too much depth away from the wall. For a wall hung sterilization box, this is the only way to sterilize two very different sized devices at the same time.

FIG. 9 Shows the Holes in the cover which enable the power cord access to the charging outlet on the outside of the sterilization box.

FIG. 10: Shows the power outlet built into the outside of the sterilization box for use in charging battery powered portable electronic devices during sterilization.

### DETAILED DESCRIPTION OF THE INVENTION

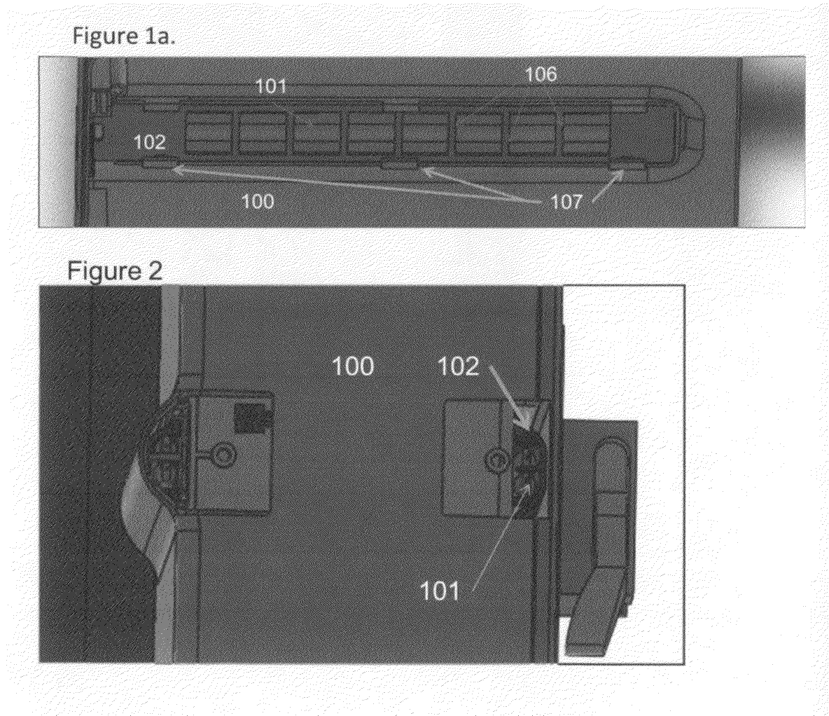

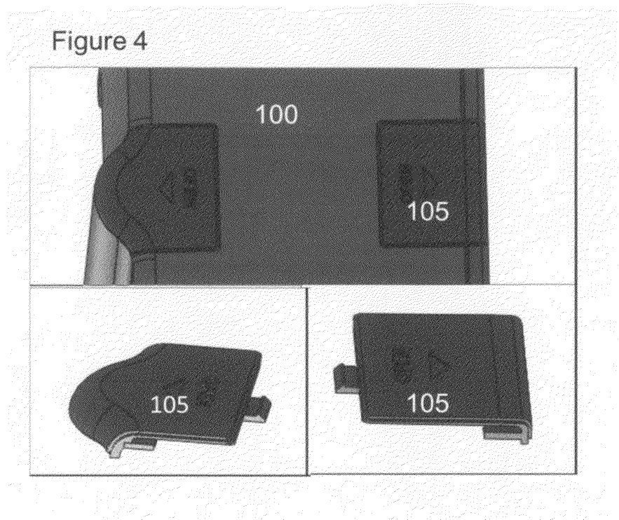

As mentioned above in this disclosure UVC bulbs that need to fit into smaller boxes for sterilization applications are typically cold cathode UVC bulbs. These bulbs are very small and are very fragile. Their performance is also lessened if hand prints get on the glass part of the bulb, so you do not want human hands with all of the grease and etc. on them to touch the cold cathode UVC bulb during the bulb installation process. We created a separate “package” or enclosure for this UVC bulb 102 which provides the following combined functions for its use inside a UVC sterilization box:
Protects the cold cathode UVC bulb 101 when it is installed.
Ensures no finger prints get on the cold cathode UVC bulb 101 when it is installed.
Provides reflective surface for the UVC bulb 101 when it is in operation inside the sterilization box. The rear side of the plastic package is metalized so that it will reflect all of the light back into the sterilization box
Provides complete protection for the cold cathode UVC bulb 101 during the operation of the sterilization box. The front part of the two part plastic assembly package 102 provides this protection and is shaped like an open “grill” 106. This grill design is shown clearly in FIG. 1 and in FIG. 4. As a result of this grill design this allows all of the UVC light from the bulb to be provided into the sterilization box without the items that are being put into the box being able to break or crack the cold cathode UVC bulb 101.

To make the installation of this cold cathode uvc bulb assembly 102 very easy for the user there are slide rails 107 build into the inside of the sterilization box 100. These rails 107 can be seen clearly in FIG. 1

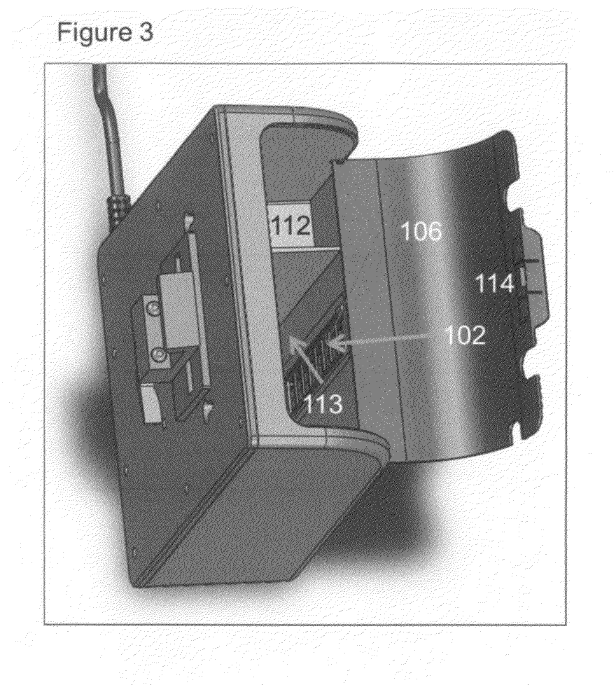

FIG. 2 shows how the two part plastic UVC bulb assembly 102 slides into the sterilization box 100 from the bottom. Those are channels built into the sterilization box to accept this uvc bulb enclosure.

FIG. 4 shows the last part of this unique solution where a simple snap fit cap 105 is placed over the top of the UVC bulb channel opening on the sterilization box 100 (these channels are shown clearly in FIG. 2. These snap fit caps can be designed to be part of the sterilization enclosure 100 or the UVC bulb enclosure 101. We created them to be part of the overall sterilization enclosure because we did not want to have to replace them with each change out of the UVC bulb 101.

It is important to note that with this design no expensive and bulky UVC bulb socket is required inside the sterilization box 100 and no UVC bulb base is required for the cold cathode UVC bulb 101. The only thing required is a simple wire with low cost $0.01 male connector on the UVC bulb 101 and a female connector on the Sterilization enclosure that it snaps into.

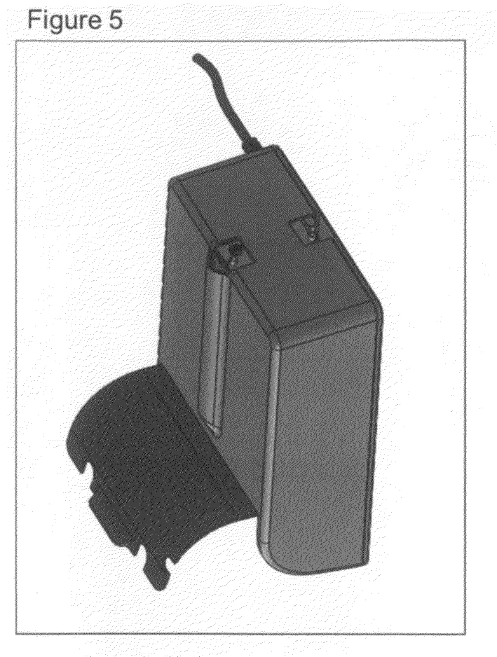

FIG. 5 provides another view of the bulb assembly 102 slid and snapped into the Sterilization box 100 from the bottom side up.

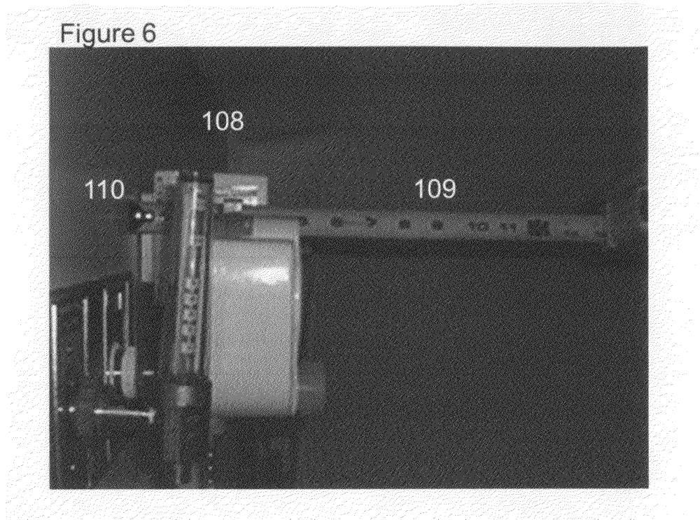

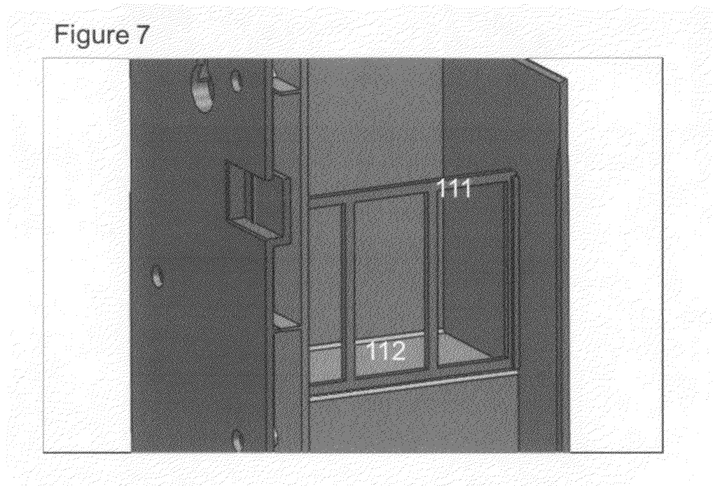

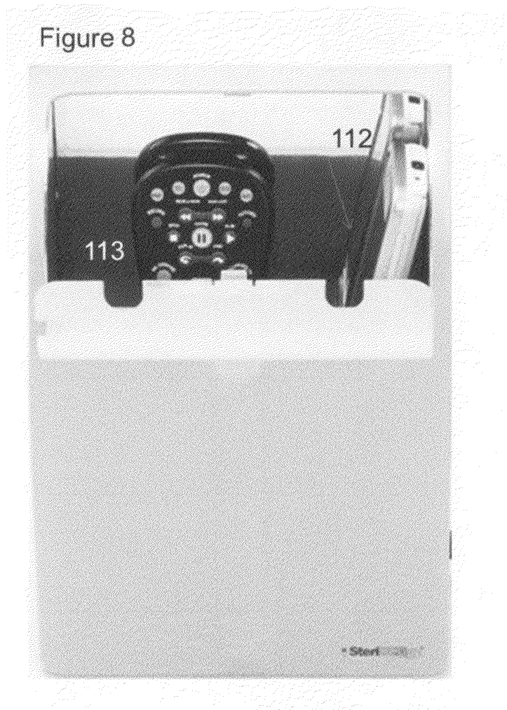

FIG. 6 shows the environment inside the hospital room. The wall of the hospital patient room 110 next to the patient bed right next to the bed of the patient has many devices hanging off of it for patient care 108. None of these devices 108 can protrude off of the wall 110 by more than 5-6 inches when measured by a standard ruler 109. If they protrude off the wall by more than 6 inches then they will be knocked off by the patient or nurse or doctor when they are servicing the patient. Because a sterilization box 100 for remote controls would need to be on this wall 110 so that it would be access able for the patient to place their electronic device items into, the depth of the sterilization box will need to be small. This creates a major challenge for enabling the patient to put both small and large electronic devices into the box AND get them back out of the box when they are done. We solved this problem by coming up with a sterilization box 100 that has two distinct levels built into it 112 and 113 so that smaller devices like mobile phones and car keys could be placed on the higher level 112 and larger devices like corded phones and remote controls and e book readers could be placed on the lower level 113 where there is more depth and more room. To enable the UVC light to get access to all areas of the UVC sterilization box 100 including the top level 112 we designed grill based bracket 111 between the upper level 112 and lower level 113. This grill bracket 111 is critical because it holds the smaller electronic devices in place so that they do not slip down into the lower level 113. If they slip down into this level then are almost impossible for the patient to get back out. FIG. 7 shows how the two types of devices (large and small) can sit right next to each other in this very small depth sterilization box with the top sided cover or opening and closing and shows how they are both easily accessible by the user. FIG. 3 shows the last part of the solution which enables this very small depth UVC sterilization box 100 which hang off the wall or cabinet to sterilize both large and small devices with the utmost convenience. The box must open from the top so that the patient can place the devices into the sterilization box without them slipping back out again before the door closes. The devices must stand up vertically to be sterilized in this type of box (so that they fit in) so you cannot have the door on the front of the sterilization box 100. We placed the door 114 on the top but the door hinges at the middle of the front of the box so that the inside of the box is more accessible and so that the two levels shown in practical use in FIG. 8 can be accessed with ease by the patient from the hospital bed.

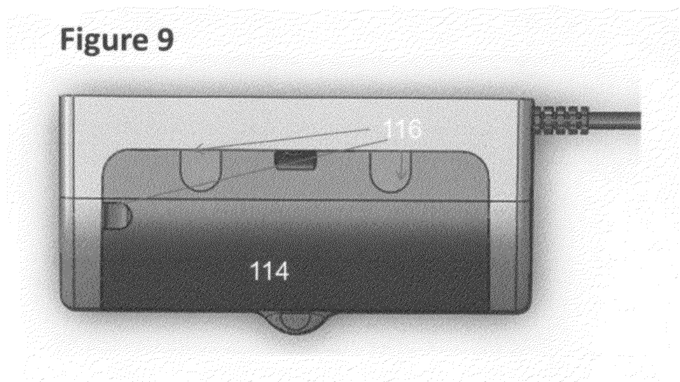

FIG. 9 is another view of the door 114 to the sterilization box 100. You can see that the door closes and snaps fits shut into the top of the unit but it hinges at the middle of the front of the box so that the two levels 112 and 113 are completely and easily accessible.

Now you can also see from FIG. 9 that this same cover has holes 116 built into it so that it can sterilize corded devices like hospital remote controls that include speakers for the TV and are powered and connected off the hospital room wall 110. These holes 116 also allow for the electronic devices that are being sterilized inside the unit to all be charged by a build in charging outlet on the unit itself. You can see this charging outlet 120 on the side of the sterilization unit 100. This charging outlet 120 has a removable cover on it to keep it clean when not in use.

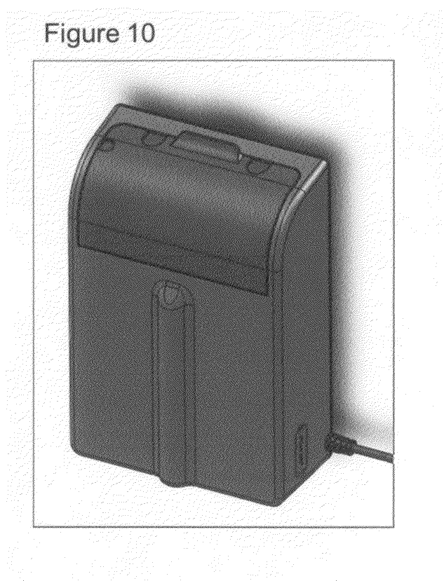

The FIG. 10 shows the outlet with the cover in place. There are no directly reachable electrical outlets on the wall of the hospital 110 by the patient for themselves. When they want to plug in their portable device then need to call for the nurse or somebody on the hospital staff to help them out with this. This outlet now gives the patient another reason to put their portable devices inside the sterilization unit for cleaning because they can also charge their device at the same time. This drives compliance with the cleaning routines of the hospital where hospital acquired infections are such a problem.

I claim:

1. A UV sterilization or sanitizing device comprising:
A power supply, multiple germicidal lamps, a housing to hold the devices being sterilized and a cover with various sized reliefs built into it to allow for power cords to pass through it so that wired electronic devices may be sterilized without transmitting ozone into the immediate environment or allow germicidal light to come into contact with human eyes.

2. The UV sterilization or sanitizing device according to claim 1 wherein multiple levels are built into the interior housing of the sterilization device to allow for different sized devices sterilized effectively.

3. The UV sterilization or sanitizing device according to claim 1 wherein one of the reliefs built into the cover allow for a standard charging device wire to pass through it for the purpose of charging an electronic device while it is inside the sterilization device.

4. A UV sterilization or sanitizing device with replaceable UV lamps which do not require humans to directly contact the UV lamps when replacing them comprised of:
cold cathode germicidal lamps
a two part enclosure which assembles together around the UV lamps;
a channel or relief built into the sterilization device housing to fit the two part enclosure with the cold cathode UV lamps inside

[symbol]

5. The UV sterilization or sanitizing device with replaceable UVC lamps according to claim 4 wherein the connection between the sterilization device and the replaceable UVC lamps is a wire harness.

6. The UV sterilization or sanitizing device with replaceable UVC lamps according to claim 4 where the two part plastic enclosure which snaps together around the lamps is metalized plastic material.

7. The UV sterilization or sanitizing device with replaceable UVC lamps according to claim 4 where the channel or relief that the replaceable UVC lamps are inserted into is covered with removable and replaceable covers.

8. A UV sterilization or sanitizing device with a built in power source such that an electronic device can be charged while it is being sterilized.

9. The UV sterilization or sanitizing device according to claim 8 wherein the electronic device being sterilized can be charged through the sterilization device's power supply and embedded power connector using the electronic devices' standard power supply while it is being [symbol]
